(19) United States
(12) Patent Application Publication
(10) Pub. No.: US 2023/0092706 A1
(43) Pub. Date: Mar. 23, 2023

(54) TREATMENT OF VIRAL PATHOLOGIES WITH EXOGENOUS KETONES

(71) Applicant: Buck Institute for Research on Aging, Novato, CA (US)

(72) Inventors: Brianna J. Stubbs, Richmond, CA (US); John C. Newman, San Francisco, CA (US)

(73) Assignee: Buck Institute for Research on Aging, Novato, CA (US)

(21) Appl. No.: 17/918,542

(22) PCT Filed: Apr. 13, 2021
(86) PCT No.: PCT/US2021/027121

§ 371 Date: Oct. 12, 2022

Related U.S. Application Data
(60) Provisional application No. 63/011,199, filed on Apr. 16, 2020.

(51) Int. Cl. A61K 31/19; A61K 31/047; A61K 31/22; A61K 31/198; A23L 33/10

(52) U.S. Cl. CPC A61K 31/19; A61K 31/047; A61K 31/22; A61K 31/198; A23L 33/10; A23V 2002/00

(57) ABSTRACT

Provided herein are methods for the treatment and/or prophylaxis of a viral pathology in a subject using one or more exogenous ketone(s).

### CROSS-REFERENCE TO RELATED APPLICATIONS

This application claims the benefit of priority to U.S. Provisional Application Ser. No. 63/011,199 filed on Apr. 16, 2020, the entire contents of which are incorporated by reference herein.

### FIELD OF THE INVENTION

The field of the invention relates to medicine. More specifically, the present disclosure relates to treatment and prophylaxis of viral pathologies with exogenous ketones.

### BACKGROUND

Emerging viral infections continue to pose a major threat to global public health, the most common of which are those that initially infect the upper respiratory system. For example, viral infections by influenza viruses (A and B), H5N1 and H7N9 avian influenza A viruses, parainfluenza viruses 1-4, adenoviruses, respiratory syncytial virus A and B and human metapneumovirus, and rhinoviruses commonly cause such respiratory pathologies.

In the first decade of 2000, several coronaviruses have been identified that cause a respiratory infection that can be severe and fatal. In 2002 and 2003, an outbreak of severe acute respiratory syndrome (SARS) caused a number of deaths in China and Hong Kong. In 2012, a novel coronavirus appeared in Saudi Arabia, Middle East respiratory syndrome coronavirus (MERS-CoV), which can cause severe acute respiratory illness and is sometimes fatal.

More recently, in 2019, another coronavirus (SARS-CoV2) that can cause an acute, sometimes fatal respiratory illness (COVID-19) emerged in Wuhan, China and is currently spreading worldwide. This virus has caused a global pandemic that has infected over 115 million people, killed over 2.5 million (as of March 2021) and led to worldwide social and economic disruption.

Several population sub-groups appear to be at particularly high-risk of infection related complication. First, older adults have increased risk of hospitalization and mortality when infected with COVID-19 (Team MMWR Morb Mortal Wkly Rep. 2020. 69(12): p. 343-346). Second, patients with diabetes (Type 1 and Type 2) appear to be particularly vulnerable to COVID-19 (Id.). Hallmarks of these diseases are hyperglycemia and/or insulin dysregulation, both historically shown to worsen infection risk in this population (Simonsen et al., BMJ Open Diabetes Res Care, 2015. 3(1): p. e000067; Livingstone et al., JAMA, 2015. 313(1): p. 37-44; Conway et al., Diabet Med, 2018. 35(6): p. 729-736; and McAlister et al., Diabetes Care, 2005. 28(4): p. 810-5). COVID-19 can lead to acute respiratory distress syndrome (ARDS) (Wu et al., JAMA Int. Med., 2020) and death from respiratory failure. ARDS is a complex syndrome that initiates with lung vascular endothelial injury and alveolar epithelial injury and is characterized histologically by the filling of alveoli with protein rich fluid (Fan et al. JAMA, 2018. 319(7): p. 698-710). The pathophysiology of ARDS includes complex host-pathogen immune interactions along with cellular damage and death resulting from inflammation, hyperoxia, hypoxia, and oxidative stress (Matthay et al., Nat. Rev. Dis. Primers, 2019. 5(1): p. 18). Thus, there is an urgent need to aid in the recovery in the progressive COVID-19 pandemic and well as in other severe viral pathologies.

There is a critical need for mitigation strategies that inhibit these damage pathways caused by viral infections. Even in patients who recover from critical illness and ARDS, there are long term impacts to health from prolonged critical illness that correlate with age (Azoulay et al., Crit. Care, 2017. 21(1): p. 296 and Sasannejad et al., Crit. Care, 2019. 23(1): p. 352), reinforcing that clinical strategies should focus on not only mitigating the acute disease process, but also on supporting recovery (Brummelet et al., Am. Thorac. Soc., 2018. 15(5): p. 518-522).

Thus, what is needed are more effective methods for treating the pathologic repercussions of viral infections.

### BRIEF DESCRIPTION OF THE DRAWINGS

[Not Applicable]

### SUMMARY

It has been discovered that certain exogenous ketone are effective therapeutics for the treatment and prophylaxis of many viral pathologies. This discovery has been exploited to produce the present invention, which, in part, is directed to methods of treating and/or prophylaxis of a virial pathology.

In one aspect, provided herein is a method for the treatment and/or prophylaxis of a viral pathology in a subject, the method comprising administering to the subject an effective amount of one or more exogenous ketone(s).

In some embodiments, the one or more exogenous ketone(s) comprises (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, an ester that is 3-hydroxybutyl 3-hydroxybutyrate acetoacetate, and an ester of (R)-1,3-butanediol that is mono- or di-substituted with acetoacetate. In particular embodiments, the one or more exogenous ketone(s) is selected from the group consisting of (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, an ester that is 3-hydroxybutyl 3-hydroxybutyrate acetoacetate, and an ester of (R)-1,3-butanediol that is mono- or di-substituted with acetoacetate.

In some embodiments, the one or more exogenous ketone(s) comprises (R)-3-hydroxybutyrate. In certain embodiments, (R)-3-hydroxybutyrate is a free acid. In other embodiments, the one or more exogenous ketone(s) comprises (R)-3-hydroxybutyrate as a salt, and in specific embodiments, the salt is a mineral salt or an amino acid salt. In certain embodiments, the mineral salt is selected from the group consisting of a sodium salt, a potassium salt, a calcium salt, and a magnesium salt, and in one embodiment, the salt is a leucine salt. In other embodiments, the one or more exogenous ketone(s) comprises an ester of (R)-3-hydroxybutyrate. In some embodiments, the ester is mono- or di-substituted at the hydroxyl or carboxyl group by independently substituted or unsubstituted C(4-10) alkyl. Embodiments the alkyl group is substituted with hexanoic acid, a hydroxyl group, or an alkyl branch.

In other embodiments, the one or more exogenous ketone(s) comprises (R)-1,3-butanediol. In certain embodiments, the one or more exogenous ketone(s) comprises an ester of (R)-1,3-butanediothe, and in certain embodiments, the ester of (R)-1,3-butanediol is mono- or di-substituted at the hydroxyl or carboxyl group by independently substituted or unsubstituted C(4-10) alkyl or is mono- or di-substituted with acetoacetate. In particular embodiments the alkyl group is substituted with hexanoic acid, a hydroxyl group, or an alkyl branch.

In some embodiments, the ester is enantiomerically enriched with the R-enantiomer, an S-enantiomer, or is a racemic mixture of R- and S enantiomers.

In other embodiments, the one or more exogenous ketone(s) comprises acetoacetate.

In still other embodiments, the at least one exogenous ketone has the structure of Formula I:

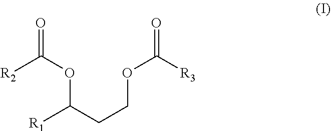

wherein R₁ is hydrogen or alkyl; and R₂ and R₃ are alkyl.

In other embodiments, the at least one ketone has the structure of Formula II:

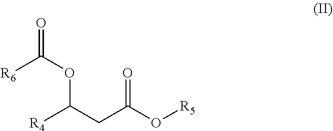

wherein R₄ is hydrogen or alkyl; and R₅ and R₆ are alkyl.

In certain embodiments, the at least one exogenous ketone is provided as a pharmaceutical formulation. a dietary supplement, or a food substance.

In some embodiments, the method further comprising administering to the subject a therapeutically effective amount of an additional agent that is not a ketone as described herein.

In some embodiments, the subject is treated for a viral pathology characterized by sudden acute respiratory syndrome (SARS), oxidative stress, tissue hypoxemia, impaired glycemic control, and/or impaired mitochondrial function. In another embodiment the viral pathology is characterized by a cytokine storm.

In certain embodiments, the subject is treated for a viral pathology is produced by a virus selected from the group consisting of coronaviruses, human influenza viruses Types A and B, human Rhinovirus, respiratory syncytial virus, parainfluenza viruses (HPIVs), measles virus, rubella virus, chickenpox/shingles virus, smallpox virus, polio virus, chikungunya virus, and hepatitis viruses. In a specific embodiment, the viral pathology is produced by a coronavirus that is a member of the Betacoronavirus Genus. In particular embodiments, the coronavirus is selected from the group consisting of that SARS-CoV-2 (2019-nCoV), SARS-CoV-2, and MERS-Co. In yet another embodiment, the coronavirus produces severe acute respiratory syndrome (SARS). In a specific embodiment, the viral pathology is COVID-19 produced by SARS-Cov-2.

In some embodiments, the subject that is treated is a mammal. In certain embodiments, the mammal is a human and in other embodiments, the mammal is a non-human mammal. In certain embodiments, the subject is diagnosed as having the viral pathology, and in other embodiments, the subject is identified as being at risk for the viral pathology. In one embodiment, the subject is a healthcare worker. In yet another embodiment, the subject is a subject in contact with other subjects having putative symptoms of the viral pathology. In still another embodiment, the subject is a subject in contact with other subjects diagnosed as having the viral pathology. In some embodiments, the subject is elderly, is over 50 years old, over 60 years old, over 70 years old, over 80 years old, or over 90 years old). In other embodiments, the subject is obese, has diabetes, and/or is diagnosed with hypertension or cardiac disease. In still other embodiments, the subject has or has previously had cancer, and in a specific embodiment, the subject has lung cancer or has previously had lung cancer. In certain embodiments, the subject has a respiratory pathology, is a smoker, has emphysema or COPD. In a specific embodiment, the subject is diagnosed as positive for COVID-19.

In another embodiment, the method further comprising administering to the subject an effective amount of an additional therapeutic agent that is not a ketone. In certain embodiments, the additional therapeutic agent is selected from the group consisting of an anti-viral monoclonal antibodies, entecavir, tenofovir, lamivudine, telbivudine, adefovir, clevudine, hexadecyloxypropyl tenofovir, AGX-1009, zidovudine, didanosine, zalcitabine, stavudine, emtricitabine, abacavir, reverset, remdesivir, alovudine, amdoxovir, elvucitabine, delavirdine, efavirenz, nevirapine, capravirine, calanolide A, rilpivirine, BMS-561390, DPC-083, molnupiravir, PF-07321332, prodrugs thereof, pharmaceutically acceptable salts thereof, and combinations thereof.

In another aspect, the disclosure provides a composition comprising one or more exogenous ketone(s) in a therapeutically effective amount for the treatment or prophylaxis of a viral pathology in a subject. In some embodiments, the one or more exogenous ketone(s) comprises (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, 3-hydroxybutyl 3-hydroxybutyrate, acetoacetate, or an ester of (R)-1,3-butanediol that is mono- or di-substituted with acetoacetate.

In yet another aspect, the disclosure provides use of one or more exogenous ketone(s) in the manufacture of a medicament for the treatment or prophylaxis of a viral pathology in a subject. In some embodiments, the one or more exogenous ketone(s) comprises (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, 3-hydroxybutyl 3-hydroxybutyrate, acetoacetate, or an ester of (R)-1,3-butanediol that is mono- or di-substituted with acetoacetate.

### DESCRIPTION

The disclosures of these patents, patent applications, and publications in their entireties are hereby incorporated by reference into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “treat,” “treated,” “treating,” or “treatment” includes the diminishment or alleviation of at least one symptom associated or caused by the viral pathology being treated.

As used herein, the term “prevent” or “prevention” means no infection, disorder, or pathological development if none had occurred, or no further disorder or pathological development if there had already been development of the disorder or infection. Also considered is the ability of one to prevent or reduce some or all of the symptoms associated with the disorder or disease.

As used herein, the terms “subject,” “patient,” or “individual” refers to a human or a non-human mammal. Non-human mammals include, but are not limited to, livestock and pets, such as ovine, bovine, porcine, canine, feline, murine, and marine mammals.

As used herein, the terms “effective amount,” “pharmaceutically effective amount,” and “therapeutically effective amount” refer to a nontoxic but sufficient amount of an exogenous ketone or other therapeutic agent to provide the desired biological result, such as a reduction or alleviation of the signs, symptoms, or causes of a viral infection, or any other related and desired alteration of a biological system. An appropriate therapeutic amount in any individual case may be determined by one of ordinary skill in the art using routine experimentation.

As used herein, the term “viral pathology” refers to the symptomatic or other problematic repercussions of a viral infection.

As used herein, the term “pharmaceutically acceptable” refers to a material, such as a carrier or diluent, which does not abrogate the biological activity or properties of the exogenous ketone or other therapeutic agent administered as a therapeutic or prophylactic, and is relatively non-toxic, i.e., the material may be administered to an individual without causing undesirable biological effects or interacting in a deleterious manner with any of the ketones and other active components of the composition in which it is contained.

As used herein, the term “pharmaceutically acceptable salt” refers to derivatives of the disclosed ketones wherein the parent compound (ketone) is modified by converting an existing acid or base moiety to its salt form. Nonlimiting examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids.

As used herein, the term “composition” refers to the ketone in any usable form.

The term “pharmaceutical composition”, or “formulation” refers to at least one ketone and/or salt thereof, according to the disclosure in a pharmaceutically acceptable carrier, and may encompasses another or other therapeutic agents.

An “oral dosage form” includes a unit dosage form prescribed or intended for oral administration.

A “food product” as used herein is an edible material composed primarily of one or more of the macronutrients protein, carbohydrate and fat, which is used in the body of an organism (e.g., a mammal) to sustain growth, repair damage, aid vital processes or furnish energy. A food product may also contain one or more micronutrients such as vitamins or minerals, or additional dietary ingredients such as flavorants and colorants.

A “functional food” is a food that is marketed as providing a health benefit beyond that of supplying pure nutrition to the consumer. A functional food typically incorporates an ingredient such as a micronutrient as mentioned above, that confers a specific medical or physiological benefit other than a nutritional effect, and may carry a health claim on the packaging.

A “nutraceutical” is a food ingredient, food supplement or food product that is considered to provide a medical or health benefit, including the prevention and/or treatment of disease. In general, a nutraceutical is specifically adapted to confer a particular health benefit on the consumer, and may contain a micronutrient such as a vitamin, mineral, herb, and/or phytochemical at a higher level than would be found in a corresponding regular (natural) food product. That level is typically selected to optimize the intended health benefit of the nutraceutical when taken either as a single serving or as part of a diet regimen or course of nutritional therapy.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. In an embodiment, C₁-C₆ alkyl groups are provided herein. Nonlimiting examples include methyl, ethyl, propyl, isopropyl, butyl, isobutyl, tert butyl, pentyl, neopentyl, and hexyl. Other nonlimiting examples of C₁-C₆ alkyl include ethyl, methyl, isopropyl, isobutyl, n-pentyl, and n-hexyl.

As used herein, the term “substituted” means that an atom or group of atoms has replaced hydrogen as the substituent attached to another group.

The present disclosure provides therapeutic compositions, dietary supplements, and food substances comprising at least one exogenous ketone and uses thereof, including methods of treating or preventing a viral infection or the development of a viral pathology, and methods of preparing a medicament for the treatment of viral pathologies.

### Ketones

Ketones, or ketone bodies, are any of the three compounds acetoacetic acid, acetone, and beta-hydroxybutyric acid which are normal intermediates in lipid metabolism. Endogenous ketone bodies have roles in energy metabolism, inflammation, and stress resistance. Their pleotropic activities at the interface of aging, metabolism, and inflammation are useful in mitigating aspects of various viral infections and resulting pathologies, particularly among patients most susceptible to severe disease.

Undernutrition and prolonged fasting can be harmful when applied to the intensive care unit (ICU). However, exogenous sources of ketones can directly raise circulating ketone levels equivalent to ‘physiological’ levels (about 0.5 mM to about 8 mM) within minutes without dietary or pharmacologic interventions. Exogenous ketones have a strong safety profile, low risk of toxicity, and some examples are classified as “generally recognized as safe” (GRAS) by the US Food and Drug Administration.

Illustrative exogenous ketone compounds that can be used in the methods described herein, e.g., prevent or treat viral infections and accompanying pathologies include, but are not limited to, (R)-3-hydroxybutyrate, e.g., as a free acid, as a mineral salt, e.g., of sodium, potassium, calcium or magnesium, or as a salt of an amino acid, such as, but not limited to, leucine, arginine, and glycine; an ester of (R)-3-hydroxybutyrate, e.g., that is mono- or di-substituted at the hydroxyl or carboxyl-group by independently substituted or unsubstituted C(4-10) alkyl, or which is mono- or di-substituted alkyl, at the hydroxyl or carboxyl-group by independently substituted or unsubstituted C(4-10) alkyl or C(4-10 alkyl substituted with, e.g., hexanoic acid, a hydroxyl group, or an alkyl branch; (R)-1,3-butanediol or an ester of (R)-1,3-butanediol, e.g., that is mono- or di-substituted with acetoacetate, or that is mono- or di-substituted with acetoacetate; an ester such as 3-hydroxybutyl 3-hydroxybutyrate acetoacetate; an ester such as 3-hydroxybutyl 3-hydroxybutyrate that is enantiomerically enriched either with the R-enantiomer or with the S-enantiomer, or that is a racemic mixture of R- and S-enantiomers; acetoacetate; or exogenous ketones described in U.S. Pat. No. 10,562,839 which has Formula I:

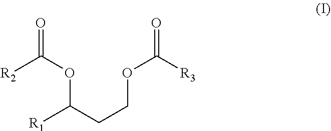

wherein R₁ is hydrogen or alkyl; and R₂ and R₃ are alkyl; and which has Formula II:

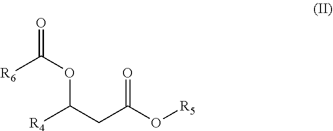

wherein R₄ is hydrogen or alkyl; and R₅ and R₆ are alkyl.

These ketones and their salts can be synthesized by any method known in the art (see, e.g., WO2017165445A1; or WO2014153416A1), or can be commercially obtained, e.g., from https://compoundsolutions.com/ingredients/bhb/; https://pruvitnow.com; https://realketones.com). For example, medium chain triglycerides are commercially available (e.g., from https://perfectketo.com). Esters containing ketones and ketone precursors are also commercially available (e.g., from https://www.tdeltas.global; https://shop.ketoneaid.com; and JuvLabs).

### Formulations

In the methods of the present disclosure, an exogenous ketone can be provided to a subject as a pharmaceutical formulation useful in treating or preventing viral infections and/or viral pathologies. Pharmaceutical formulations comprise an amount of at least one exogenous ketone described above effective to treat a subject with a viral pathology such that at least once symptom of the pathology is reduced, or effective to prevent or reduce the risk of a subject experiencing the viral pathology, and a pharmaceutically acceptable carrier.

The term “pharmaceutically acceptable carrier” is to be understood herein as referring to any substance that may, medically, be acceptably administered to a subject, together with a ketone or salt thereof, according to the disclosure, and which does not undesirably affect the pharmacological and synergistic activity of the ketone. A “pharmaceutically acceptable carrier” may thus be, for example, a pharmaceutically acceptable member(s) comprising of diluents, preservatives, solubilizers, emulsifiers, adjuvant, tonicity modifying agents, buffers as well as any other physiologically acceptable vehicle. These formulations are prepared with the pharmaceutically acceptable carrier in accordance with known techniques, for example, those described in Remington, The Science and Practice of Pharmacy (23rd Ed. (2020) Adejare, editor, Academic Press).

The exogenous ketones administered as a formulation, dietary supplement, or food substance can also be in the form of conventional, non-toxic salts which can be prepared, for example, from non-toxic inorganic or organic acids. The pharmaceutically acceptable ketone salts of the present disclosure can be synthesized from the parent ketone which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media such as, but no limited to, ether, ethyl acetate, ethanol, isopropanol, or acetonitrile are useful. The phrase “pharmaceutically acceptable salt” is not limited to a mono, or 1:1, salt. For example, “pharmaceutically acceptable salt” also includes bis-salts, such as a bis-hydrochloride salt. Lists of representative suitable salts are found in Remington's Pharmaceutical Sciences, 17th ed. (Mack Publishing Company, Easton, Pa., 1985, p. 1418) and in Berge et al. (J. Pharm. Sci. (1977) 66(2):384).

For use in medicine, the salts of the exogenous ketones are pharmaceutically acceptable salts. Other salts may, however, be useful in the preparation of the ketones or of their pharmaceutically acceptable salts according to the disclosure. Suitable pharmaceutical salts of the ketones useful in the methods according to the present disclosure include acid addition salts which may, for example, be formed by mixing a solution of a pharmaceutically acceptable acid such as hydrochloric acid, sulphuric acid, methanesulphonic acid, fumaric acid, maleic acid, succinic acid, acetic acid, benzoic acid, oxalic acid, citric acid, tartaric acid, carbonic acid or phosphoric acid. Additionally, where the ketones in the formulation carries an acidic moiety, suitable pharmaceutically acceptable salts thereof may include alkali metal salts, e.g., sodium or potassium salts; alkaline earth metal salts, e.g. calcium or magnesium salts; and salts formed with suitable organic ligands, e.g., quaternary ammonium salts.

The pharmaceutical formulations may be prepared for injectable use, topical use, oral use, intramuscular or intravenous injection, inhalation use, transdermal use, intradermal, transmembrane use, and the like. These formulations are in unit dosage forms such as tablets, pills, capsules, powders, granules, sterile parenteral solutions or suspensions, metered aerosol or liquid (nebulized) sprays, drops, ampoules, auto-injector devices or suppositories; for oral parenteral, intranasal, sublingual topical or rectal administration, or for administration by inhalation or insufflation.

A therapeutically effective dosage of the formulation according to the disclosure depends on the viral pathology being treated, and may vary from subject to subject. Factors affecting dosage include age, weight, and physical size of the subject, the subject's genetics, and the diagnosed condition of the subject. A therapeutically effective dose and frequency of administration of a dosage form may be determined in accordance with routine pharmacological procedures known to those skilled in the art. For example, dosage amounts and frequency of administration may vary or change as a function of time and severity of the disorder. A dosage from about 1.0 μg/kg to about 1000 mg/kg exogenous ketone may be suitable. For example, suitable dosage levels are from about 100 μg/kg to about 500 mg/kg, from about 1.0 mg/kg to about 750 mg/kg per day, or form about 100 mg to about 500 mg. The formulation may be administered as a bolus and/or a regimen of about 1 to about 4 times per day, or as continuous administration via enteral tube feeds. For example, for the treatment of COVID-19, a suitable dosage level is about 1.0 μg/kg to about 250 mg/kg per day. Alternatively, the formulations may be presented in a form suitable for one-weekly or once-monthly administration; for example, an insoluble salt of the derivative, such as decanoate salt, may be adapted to provide a depot preparation for intramuscular injection. An erodible polymer containing the inhibitor may also be envisaged.

For preparing solid compositions such as tablets, the one or more ketone is mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a mixture of a ketone according to the present disclosure, or a pharmaceutically acceptable salt thereof.

These formulations may be homogeneous, i.e., the ketone is dispersed evenly throughout the composition so that the composition may be readily subdivided into equally effective unit dosage forms such as tablets, pills and capsules. The tablets or pills comprising the ketone can be coated or otherwise compounded to provide a dosage form affording the advantage of prolonged action. The liquid forms in which the ketones may be incorporated for administration orally or by injection include aqueous solutions, suitably flavored syrups, aqueous or oil suspensions, and flavored emulsions with edible oils as well as elixirs and similar pharmaceutical vehicles.

Injectable dosage forms may be sterilized in a pharmaceutically acceptable fashion, for example by steam sterilization of an aqueous solution sealed in a vial under an inert gas atmosphere at 120° C. for about 15 minutes to about 20 minutes, or by sterile filtration of a solution through an about 0.2 μM or smaller pore-size filter, optionally followed by a lyophilization step, or by irradiation of a composition containing an inhibitor of the present disclosure by means of emissions from a radionuclide source.

When treating a subject with a ketone formulation, treatment may also include administering a formulation comprising more than one type of ketone, and/or a therapeutic agent other than a ketone which treats viral pathologies. The treatment may alternatively or additionally include administration of another therapeutic agent in a formulation separate from the formulation comprising the one or more exogenous ketones Useful nonlimiting therapeutic anti-viral agents include anti-viral monoclonal antibodies, entecavir, tenofovir, lamivudine, telbivudine, adefovir, clevudine, hexadecyloxypropyl tenofovir, AGX-1009, zidovudine, didanosine, zalcitabine, stavudine, emtricitabine, abacavir, reverset, remdesivir, alovudine, amdoxovir, elvucitabine, delavirdine, efavirenz, nevirapine, capravirine, molnupiravir, PF-07321332, calanolide A, rilpivirine, BMS-561390, DPC-083, prodrugs thereof, pharmaceutically acceptable salts thereof, and combinations thereof.

### Food Products and Dietary Supplements

The one or more exogenous ketones in the forms described herein may be administered as a component of, or additive to, a food product, functional food, nutraceutical, or dietary supplement in an amount effective to treat a viral pathology or to reduce the risk of a subject experiencing a viral pathology. Nonlimiting examples of food products into which the one or more exogenous ketones may be incorporated as an additive include, but are not limited, to snack bars, meal replacement bars, cereals, beverages, confectionery and probiotic formulations including, but not limited to, yoghurts. Nonlimiting examples of beverages include, but are not limited to, soft beverages, energy drinks, dry drink mixes, nutritional beverages, meal or food replacement drinks, compositions for rehydration (for instance during or after exercise), and teas (e.g., herbal teas) for infusion or herbal blends for decoction in water. Dietary supplements may be formulated as, e.g., tablets, capsules, softgels, gelcaps, powders, and liquids. When administered as a food substance, the one or more exogenous ketone is added as a solid (e.g., a powder) or liquid to an ingestible food product such as a dry good or liquid drink, baked good, fresh food such as a vegetable or fruit-containing dish, etc.

### Disorders to be Treated

Exogenous ketones are used in the treatment and prophylaxis of various viral pathologies. These viral pathologies may be characterized by sudden acute respiratory syndrome (SARS), and/or oxidative stress, and/or tissue hypoxemia, impaired glycemic control, impaired mitochondrial function, cytokine storm, and/or COVID-19. Such pathologies can be produced by infection of a coronavirus, such as but not limited to a coronavirus that is a member of the Betacoronavirus genus, such as, but not limited to, SARS-CoV-2 (2019-nCoV), SARS-CoV-2, and MERS-CoV, Infection by human influenza viruses Types A and B, a human rhinovirus, a respiratory syncytial virus (RSV), a parainfluenza viruses (HPIV), a morbillivirus (MeV) (measles) virus, a rubella virus, a varicella (chickenpox/shingles) virus, a variola (smallpox) virus, a poliovirus, a chikungunya virus, and a hepatitis virus (HAV) are also treatable by the exogenous ketones as described herein.

The presently disclosed anti-viral pathology treatments comprising administration of at least one exogenous ketone may, under some circumstances also treat other disorders if present in the subject when administered to treat the viral pathology. For example, treatment according to the disclosure may also treat cardiac pathologies, improve tolerance to tissue hypoxemia, protect against or decrease oxidative stress, reduce apoptosis, inhibit pro-inflammatory NLRP3 activation, provide anti-inflammatory effect, impair viral replication, inhibit glycolysis, and/or improve systemic glycemic control, and mitigate insulin resistance, in the subject.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A method for the treatment and/or prophylaxis of a viral pathology in a subject, the method comprising administering to the subject an amount of one or more exogenous ketone(s) effective to treat or prevent the viral pathology in the subject,
wherein the one or more exogenous ketone(s) comprises (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, 3-hydroxybutyl 3-hydroxybutyrate, acetoacetate, or an ester of (R)-1,3-butanediol that is mono- or di-substituted with acetoacetate.

2. The method of claim 1, wherein the one or more exogenous ketone(s) is selected from the group consisting of (R)-3-hydroxybutyrate as a free acid, (R)-3-hydroxybutyrate as a salt, (R)-1,3-butanediol, an ester of (R)-3-hydroxybutyrate, an ester of (R)-1,3-butanediol, 3-hydroxybutyl 3-hydroxybutyrate, acetoacetate, or an ester of (R)-1,3-butanediol that is mono- or di-substituted with acetoacetate.

3. The method of claim 1, wherein the one or more exogenous ketone(s) comprises (R)-3-hydroxybutyrate.

4. The method of claim 3, wherein the one or more exogenous ketone(s) comprises (R)-3-hydroxybutyrate as a free acid.

5. The method according to claim 3, wherein the one or more exogenous ketone(s) comprises (R)-3-hydroxybutyrate as a salt.

6. The method according to claim 5, wherein the (R)-3-hydroxybutyrate salt is a mineral salt.

7. The method according to claim 6, wherein the mineral salt selected from the group consisting of a sodium salt, a potassium salt, a calcium salt, and a magnesium salt.

8. The method according to claim 5, wherein the (R)-3-hydroxybutyrate salt is an amino acid salt.

9. The method of claim 8, wherein the amino acid is leucine.

10. The method according to claim 1, wherein the one or more exogenous ketone(s) comprises an ester of (R)-3-hydroxybutyrate

11. The method according to claim 10, wherein the one or more exogenous ketone(s) comprises an ester of (R)-3-hydroxybutyrate that is mono- or di-substituted at the hydroxyl or carboxyl group by independently substituted or unsubstituted C(4-10) alkyl.

12. The method according to claim 1, wherein the one or more exogenous ketone(s) comprises (R)-1,3-butanediol.

13. The method according to claim 1, wherein the one or more exogenous ketone(s) comprises an ester of (R)-1,3-butanediol.

14. The method according to claim 13, wherein the one or more exogenous ketone(s) comprises an ester of (R)-1,3-butanediol that is mono- or di-substituted at the hydroxyl or carboxyl group by independently substituted or unsubstituted C(4-10) alkyl.

15. The method according to claim 13 wherein the one or more exogenous ketone(s) comprises an ester of (R)-1,3-butanediol that is mono- or di-substituted with acetoacetate.

16. The method according to claim 1, wherein the one or more exogenous ketone(s) comprises 3-hydroxybutyl 3-hydroxybutyrate.

17. The method according to claim 16, wherein the one or more exogenous ketone(s) comprises 3-hydroxybutyl 3-hydroxybutyrate, that is enantiomerically enriched with the R-enantiomer.

18. The method according to claim 16, wherein the one or more exogenous ketone(s) comprises 3-hydroxybutyl 3-hydroxybutyrate, that is enantiomerically enriched with the S-enantiomer.

19. The method according to claim 1, wherein the one or more exogenous ketone(s) comprises 3-hydroxybutyl 3-hydroxybutyrate.

20. The method according to claim 19, wherein the one or more exogenous ketone(s) comprises 3-hydroxybutyl 3-hydroxybutyrate, that is a racemic mixture of R- and S enantiomers.

21. The method according to claim 1, wherein the one or more exogenous ketone(s) comprises acetoacetate.

22. The method according to any one of claims 1-21, wherein the exogenous ketone is provided as a pharmaceutical formulation.

23. The method according to any one of claims 1-21, wherein the exogenous ketone is provided as a dietary supplement or food substance.

24. The method according to any one of claims 1-23, wherein the method further comprising administering to the subject a therapeutically effective amount of an additional agent that is not a ketone.

25. The method of any one of claims 1-24, wherein the pathology is a viral pathology characterized by sudden acute respiratory syndrome (SARS), cytokine storm, oxidative stress, tissue hypoxemia, impaired glycemic control, and/or impaired mitochondrial function.

26. The method according to any one of claims 1-25, wherein the viral pathology is produced by a virus selected from the group consisting of a coronavirus, a human influenza virus Type A, a human influenza virus Type B, a human Rhinovirus, a respiratory syncytial virus, a parainfluenza virus (HPIV), a measles virus, a rubella virus, a chickenpox/shingles virus, a smallpox virus, a polio virus, a chikungunya virus, and a hepatitis virus.

27. The method of claim 26, wherein the viral pathology is produced by a coronavirus.

28. The method of claim 27, wherein the viral pathology is produced by a Betacoronavirus.

29. The method of claim 28, wherein the coronavirus produces severe acute respiratory syndrome (SARS).

30. The method of claim 29, wherein the coronavirus is selected from the group consisting of that SARS-CoV-2 (2019-nCoV), SARS-CoV-2, and MERS-CoV.

31. The method of claim 30, wherein the viral pathology is COVID-19 produced by SARS-Cov-2.

32. The method according to any one of claims 1-31, wherein the subject is a mammal.

33. The method according to any one of claims 1-31, wherein the subject diagnosed as having the viral pathology.

34. The method of claim 33, wherein the subject is diagnosed as positive for COVID-19.

35. The method according to any one of claim 1-32, wherein the subject at risk for the viral pathology.

36. The method according to any one of claims 1-35, wherein the subject is over 60 years old.

37. The method according to any one of claims 1-6 wherein the subject has hypertension, cardiac disease, a respiratory pathology, emphysema, COPD, diabetes, and/or is obese.

38. The method according to any one of claims 1Error! Reference source not found., wherein the subject has or has previously had cancer

39. The method of claim 38, wherein the cancer is lung cancer.

40. The method according to any one of claims 1-39, wherein the subject is a smoker.
